(19) United States
(12) Patent Application Publication
(10) Pub. No.: US 2022/0105241 A1
(43) Pub. Date: Apr. 7, 2022

(54) FLUORAPATITE COATED IMPLANTS AND RELATED METHODS REGARDING FEDERALLY SPONSORED RESEARCH

(71) Applicant: UNIVERSITY OF UTAH RESEARCH FOUNDATION, Salt Lake City, UT (US)

(72) Inventors: Sujeevini Jeyapalina, Salt Lake City, UT (US); James Beck, Salt Lake City, UT (US); Kent Bachus, Salt Lake City, UT (US); John Colombo, Salt Lake City, UT (US); Brian Bennett, Salt Lake City, UT (US); Kongnara Papangkorn, Salt Lake City, UT (US)

(21) Appl. No.: 17/420,579

(22) PCT Filed: Jan. 9, 2020
(86) PCT No.: PCT/US2020/012888

§ 371 Date: Jul. 2, 2021

Related U.S. Application Data
(60) Provisional application No. 62/790,642, filed on Jan. 10, 2019.

(51) Int. Cl. A61L 27/32; A61L 27/06; A61L 27/56; A61L 27/54

(52) U.S. Cl. CPC A61L 27/32; A61L 27/06; A61L 2420/02; A61L 27/54; A61L 2430/38; A61L 27/56

(57) ABSTRACT

Embodiments disclosed herein relates articles at least partially coated with fluorapatites to reduce downgrowth as well as methods of making and using the same.

### CROSS-REFERENCE TO RELATED APPLICATIONS

This application claims priority to U.S. Patent Application No. 62/790,642 filed on 10 Jan. 2019, the disclosure of which is incorporated herein, in its entirety, by this reference.

### STATEMENT REGARDING FEDERALLY SPONSORED RESEARCH

This invention was made with government support under Grant Number W81XWH-15-1-0682 (OR140116) awarded by the U.S. Department of Defense. The U.S. government has certain rights in the invention.

### BACKGROUND

Implantable devices may be surgically implanted into tissue(s) of a subject, such as a human or animal. Implantable devices often fail because the implant site becomes infected.

Human or animal subjects reject foreign objects implanted therein, because the tissues of the subject around the implantation site reject the implant. For example, the tissues around an implantation site may not integrate with the material(s) of the implantable device. Some materials may be rejected by tissues more readily than others.

### SUMMARY

Embodiments disclosed herein relate to articles at least partially coated with fluorapatites to reduce downgrowth as well as methods of making and using the same. In an embodiment, an implant is disclosed. The implant includes an implant body defining one or more surfaces. The implant includes a fluoridated apatite coating disposed on at least a portion of the one or more surfaces. The fluoridated apatite coating exhibits a surface morphology and porosity consistent with having been sintered at about 950° C. or more.

In an embodiment, a method of forming a coated implant is disclosed. The method includes providing fluoridated apatite particles that exhibit a surface morphology and porosity consistent with having been sintered at a temperature of at least 950° C. The method includes affixing the fluoridated apatite particles onto at least a portion of an implant.

In an embodiment, a method of using an implant having a fluoridated apatite coating is disclosed. The method includes providing an implant including one or more surfaces at least partially coated with fluoridated apatite, wherein the fluoridated apatite exhibits a surface morphology and porosity consistent with having been sintered at about 950° C. or more. The method further includes implanting the implant in a subject.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the invention, wherein identical reference numerals refer to identical or similar elements or features in different views or embodiments shown in the drawings.

FIG. 1 is a side cross-sectional view of an uncoated implant disposed in a wound site, according to an embodiment.

FIG. 2A is a side cross-sectional view of a coated implant disposed in a wound site, according to an embodiment.

FIG. 2B is a side cross-sectional view of a coated implant, according to an embodiment.

FIGS. 3A-3D are electron photomicrographs of fluorapatite before and after sintering at 1,200° C.

FIG. 4 is a flow diagram of a method of making a coated implant, according to an embodiment.

FIGS. 5A-5D are electron photomicrographs of a fluorapatite coating on an implant

FIG. 6 is a bar chart showing the enumerated adherent HaCaT cells, from each sample surface two days after seeding.

FIG. 7 is a bar chart showing the enumerated Involucrin-positive nuclei on the respective samples.

FIG. 8 is a flow chart of a method of using an implant having a fluoridated apatite coating, according to an embodiment.

FIGS. 9A-9D are photographs of tissue sections for histology analysis of implanted titanium, HA, FFA, and FA pellets, according to an embodiment.

FIGS. 10A-10D are photos of stained sections of samples showing epithelial downgrowth along the implant surfaces, according to embodiments.

FIG. 11 is a graph of quantitative values for epithelial downgrowth observed along the various implants of Groups 1-4, according to embodiments.

FIG. 12 is a graph of the granulation tissue area observed on the various implant surfaces, according to an embodiment.

### DETAILED DESCRIPTION

Embodiments disclosed herein relate to implants at least partially coated with fluoridated apatites to reduce downgrowth as well as methods of making and using the same. The fluoridated apatites disclosed herein include one or more of fluorohydroxyapatite (“FHA”) or fluorapatite (“FA”), which is sintered at a sintering temperature selected to provide a desired surface morphology for the coating. FHA (Ca₁₀(PO₄)₆ F_{y}(OH)_{2-y}) and FA (Ca₁₀(PO₄)₆F₂) are partially and fully fluoridated forms of apatite, respectively. The fluoridated apatite coatings disclosed herein demonstrate excellent adhesion to tissue cells and reduced downgrowth relative to non-coated implants or implants that have other coatings (e.g., a hydroxyapatite (“HA”) coating). The coated implants disclosed herein include percutaneous devices.

Percutaneous devices are made of nonbiological materials (e.g., materials foreign to the body's internal environment such as titanium), which penetrate to a deep attachment point, often in bone, through the skin to provide a link between an internal organ or cavity and an external device. Percutaneous devices are used in a multitude of medical applications. Dental implants, bone-anchored hearing aids (BAHAs), percutaneous osseointegrated prosthetic skeletal docking devices for attachment of artificial limbs, catheters, and feeding tubes, are just a few examples of these devices. Due to failure of the soft tissue interface to permanently attach to the surface of these devices, stomal healing is incomplete, leaving the implant/skin interface prone to infection. For example, immediately following a percutaneous implantation, phenotypically changed epithelial cells migrate proximally along the implant surface in an attempt to seek a biological signal from their counterparts to complete the healing cascades. The presence of a percutaneous device may essentially block response of normal wound healing cascades, effectively causing skin downgrowth and an ‘externalization’ of the implant. The phenomenon of “epithelial downgrowth” produces a sinus tract around the implant extending to the outside with downgrowth, thereby presenting a nidus for external bacterial colonization.

Percutaneous implant sites, regardless of whether they are anchored in soft tissue or bone, present a discontinuity between the deep dermal layers and the epidermis. Because the epidermis provides a physical as well as a physiological barrier between the deep biological environment (bone, muscle, and subcutaneous tissues) and the external environment, any permanent discontinuity in this barrier, without a biological attachment or seal, sooner or later leads to infection, inflammation, and ultimately, device failure. This interface region is where the external and internal environments and the implant surface meet, and is the site of invasion by pathogens. Epithelial cells do not permanently integrate with (e.g., attach with) any known implant surface, accounting for undesirable clinical outcomes such as infection and implant failure. The coatings, coated devices, and methods disclosed herein provide a readily integratable surface for attachment to the skin.

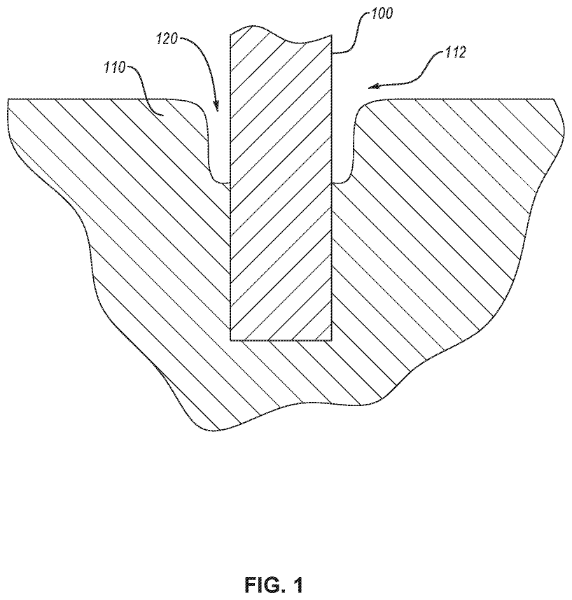

FIG. 1 is a side cross-sectional view of an uncoated implant 100 disposed in a wound site 112, according to an embodiment. The implant 100 may be disposed in tissue 110 of a wound site 112 (e.g., implantation site). The tissue 110 may include one or more tissues, such as soft tissue (e.g., skin), hard tissue (e.g., bone), or combinations thereof. A sinus tract 120 extends into the tissue 110 at the wound site 112. The sinus tract 120 may be identified as a gap between the tissue 110 and the implant 100. As the sinus tract 120 is occupied by bacteria, the bacteria may grow and cause infection(s) which may require removal of the implant 100.

The coated implants disclosed herein reduce or eliminate downgrowth of epithelial cells, osteoblast cells, or other local cells along the implant surface relative to uncoated implants 100 or implants that do not have the fluoridated apatite coatings disclosed herein. Epithelial cells showed an enhanced affinity for fluoridated apatite surfaces that were sintered at 1050° C. to 1250° C. when compared to titanium (Ti) and HA surfaces.

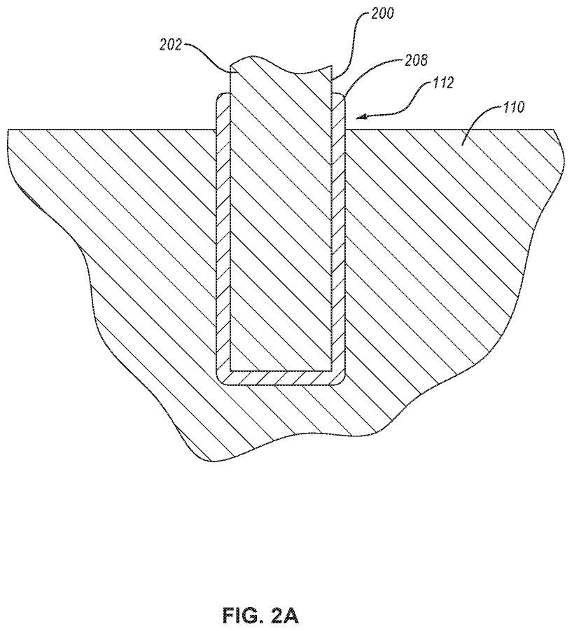

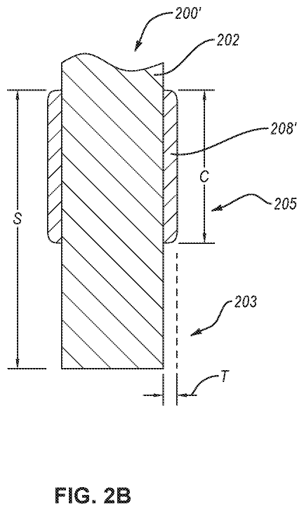

FIG. 2A is a side cross-sectional view of a coated implant 200 disposed in a wound site 112, according to an embodiment. The implant 200 may be disposed in the tissue 110 at the wound site 112. The implant 200 includes an implant body 202 and a coating 208. The implant body 202 may be formed in any suitable shape (e.g., size and dimensions) for implantation into the tissue of a subject. The implant body 202 may include metals or alloys (e.g., titanium or titanium alloys), ceramics, polymer(s) (e.g., polyether ether ketone), or any other material composed to be implanted in tissue of a subject. Such materials may be composed to be substantially inert in the tissue. For example, the material may not cause adverse reactions (e.g., toxicity) with the tissue of the subject. The implant body 202 may include one or more of a post, a rod, a plate, a screw, a pump, a drug delivery device, a wire, a tube, a joint, a socket, a ball, an electrical stimulation device, a battery, or the like. In embodiments, the implant body 202 may include a percutaneous implant such as percutaneous osseointegrated (01) prosthetics, dental implants, orthopedic implants, or the like. The implant body 202 defines a plurality of surfaces. The coating 208 can be disposed on (e.g., affixed to) at least a portion of one or more surfaces of the implant body 202.

As explained in more detail below the coating 208 may reduce or eliminate sinus tract 120 (FIG. 1) formation. Accordingly, the coated implants disclosed herein may reduce or eliminate infections at implant sites and by extension failure of implants.

As depicted in FIG. 2A, the coating 208 may be disposed on substantially all of the surfaces of the implant body 202 that are implanted in tissue(s) 110. By coating substantially all of the surfaces of the implant body 202 expected to be implanted in the tissue 210 with the coating 208, the coated implant 200 provides a medium for preferential attachment of tissue cells (e.g., epithelial cells, osteoblast cells, keratinocyte cells, etc.) to the coated implant 200. The coating 208 includes fluoridated apatite material, such as FHA, FA, or combinations thereof. FA has proven to be particularly effective at adhering to the epithelial cells. The coating 208 promotes tissue adhesion which reduces or eliminates downgrowth along the coated implant 200, thereby reducing or eliminating infections at the wound site 112 (e.g., implant site).

In some embodiments, less than entire side surface of a coated implant or only a portion of a side surface (e.g., a portion positioned to be at the surface of the skin of the implantation subject after implantation) may be coated with the fluoridated apatite coating. FIG. 2B is a side cross-sectional view of a coated implant 200′, according to an embodiment. The coated implant 200′ includes the implant body 202 and the coating 208′. The coating 208′ may be similar or identical to the coating 208 in one or more aspects. The coating 208′ may not cover all of the surfaces that may be implanted in a subject. For example, a proximal portion 203 (e.g., the end) of the implant body 202 may not be coated with the coating 208′, while a medial portion 205 is coated with the coating 208′. A total length S of the outer surface of the implant body 202 that is configured to be implanted in the tissue of a subject may be greater than the length C of the coating 208′ disposed on the outer surface. The length S may be at least about 1 mm, such as about 1 mm to about 25 cm, 1 cm to about 10 cm, about 10 cm to about 20 cm, less than about 25 cm, less than about 10 cm, or less than about 5 cm. The length C may be at least 1 mm, such as about 1 mm to about 25 cm, about 1 mm to about 5 cm, about 1 cm to about 10 cm, about 10 cm to about 20 cm, less than about 20 cm, less than about 10 cm, or less than about 5 cm. The length C may be less than one half of the length S, such as one tenth to one half of the length S, one tenth to one quarter of the length S, one quarter to one half of the length S, more than one tenth, or more than one eighth of the length S. In such embodiments, the coating 208′ may be disposed on the portion of the implant body 202 that is expected to rest at the skin surface of the subject (e.g., such that the coating 208′ extends into and out of the skin of the subject). The coating 208′ may be disposed on more than one portion of the implant 200′. For example, an implant may be configured to protrude through the skin of a subject at more than one point thereon and the implant includes the coating 208′ may be disposed on each portion of the implant that will be positioned at and around the skin of the subject. The selectively positioned coating 208′ may promote adhesion of tissue to the implant 200′ at the skin surface and to a point therebelow that is selected to prevent bacteria from penetrating into the implantation wound.

In some embodiments, the thickness T of the coating 208′ (or 208) may be at least about 1 μm, such as about 1 μm to 20 mm, about 10 μm to 10 mm, about 20 μm to 5 mm, about 100 μm to 2 mm, about 1 μm to about 1 mm, about 1 μm to about 500 μm, about 1 μm to about 50 μm, about 5 μm to about 15 μm, about 20 μm to about 300 μm, less than about 50 mm, less than about 20 mm, less than about 10 mm, less than about 5 mm, less than about 1 mm, less than 500 μm, or less than 100 μm.

In some embodiments, the thickness T of the coating 208′ (or 208) may vary in different regions of the surface of the coated implant. For example, it may be desirable to coat the medial portion 205 of the surface of the implant with a thicker portion of coating 208′ (or 208) then at a proximal portion 203 such as to encourage more tissue adhesion at the portion of the implant expected to be at the skin surface after implantation than at the proximal portion 203 which is disposed deeper within the tissue of the subject. In other embodiments, the coating 208′ (or 208) at the proximal portion 203 may have a greater thickness T than at the medial portion 205. The thickness T of one or more of the medial portion 205, the proximal portion 203, or a distal portion (not shown) of the implant surface may be, independently, any of the coating thicknesses disclosed above.

The coating 208′ (or 208) disclosed herein includes fluoridated apatite that has been sintered at a temperature between about 950° C. and about 1,350° C., or more particularly between about 1,050° C. and about 1250° C. The inventors currently believe that fluoridated apatite sintered in the temperature range(s) disclosed above agglomerate to form a plurality of bonded agglomerations of fluoridated apatite that have a size, shape, and zeta potential that encourage adhesion between tissue cells and the fluoridated apatite (e.g., FA) in the coating 208′ (or 208).

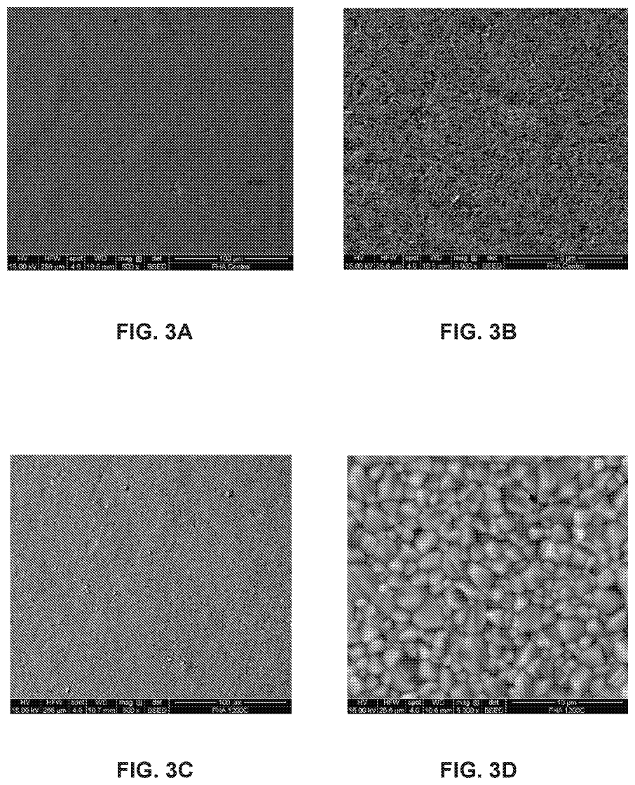

In an unsintered state, fluoridated apatite exhibits a substantially rod-like or needle-like crystal structure. During sintering, the individual fluoridated apatite crystals agglomerate and exhibit various bulk structures and surface morphologies. FIGS. 3A-3D are electron photomicrographs of FA before and after sintering at 1,200° C. FIG. 3A is an electron photomicrograph of unsintered FA shown in a first magnification (500×). As shown, the bulk structure of the unsintered FA appears to be a porous mass of particles. FIG. 3B is an electron photomicrograph of unsintered FA at a second magnification (5000×). As shown in FIG. 3B, the microstructure (e.g., each particle of the bulk structure) of the unsintered FA is rod-like or linear crystals.

The FA particles of FIGS. 3A and 3B were sintered at 1,200° C. and examined at the same magnification as FIGS. 3A and 3B to produce FIGS. 3C and 3D. FIG. 3C is an electron photomicrograph of the sintered FA shown at the first magnification. As shown, the bulk structure of the sintered FA appears to be a porous mass of particles having a greater (average) particle size than the unsintered FA shown in FIG. 3A. FIG. 3D is an electron photomicrograph of sintered FA at the magnification (5000×). As shown, the microstructure (e.g., each particle of the bulk structure) of the unsintered FA appears to be substantially granular agglomerations with a far greater average particle size (e.g., volume) than the unsintered FA particles.

FIGS. 3A-3D demonstrate that unsintered FA particles may begin with rod-like or substantially linear structure having a width (smallest dimension) of less than about 1 μm, such as less than about 0.5 μm and a length that is less than about 4 μm or less than about 2 μm; and through sintering may be formed into agglomerates exhibiting greater three dimensional characteristics. For example, the as-sintered FA (or FHA) may be agglomerated into substantially granular shapes (e.g., prismatic, pseudo-prismatic, rounded, spherical, semi-spherical, ellipsoid, or irregularly rounded shapes). The as-sintered FA (or FHA) may be substantially devoid of the rod-like or needle-like fluoridated apatite of the unsintered FA (or FHA). The average volume of an average sintered FA agglomerate may be at least ten times the average volume of the average unsintered FA particle. The smallest dimension of the average agglomerate of sintered FA particles may be at least about 0.5 μm, such as about 0.5 μm to 10 μm, or about 1 μm to 5 μm. As shown in FIGS. 3A-3D, the surface morphology of the FA particles drastically changes as a result of sintering. The resulting sintered FA particles (e.g., agglomerates), exhibit an overall smoother surface morphology than the unsintered FA particles.

Bulk fluoridated apatite particles may be a coherent mass of agglomerations provided in a specific form, such as grains. Bulk fluoridated apatite particles may be formed by sintering a mass of fluoridated apatite particles and then grinding, crushing, or otherwise breaking the resulting sintered bulk body into smaller bulk particles. The smaller bulk particles may be sized, such as using a sieve, to provide a plurality of particles having a substantially homogenous average particle size. The bulk particle size (e.g., a coherent mass of agglomerations provided in a granular form) of the bulk fluoridated apatite particles disclosed herein may be at least about 5 μm, such as about 30 μm to 300 μm, about 60 μm to 200 μm, about 65 μm to 150 μm, about 60 μm to 120 μm, about 120 μm to 200 μm, or less than about 300 μm.

The porosity of the bulk structure of the sintered FA particles is also different than the porosity of the bulk structure of the unsintered FA particles. For example, the bulk structure of the sintered FA particles exhibits less porosity than the unsintered FA particles. This is currently believed by the inventors to be due to the agglomerates densifying (e.g., self-organizing or building into naturally fitting structures) during sintering, thereby providing less pore space therebetween than the unsintered particles.

The inventors currently believe that the porosity and surface morphology of the sintered FA particles increase adhesion to tissue cells (e.g., epithelial cells, osteoblasts, fibroblasts, etc.). The charge of the fluoridated apatite material also contributes to increased tissue adhesion. For example, the surface charge of the fluoridated apatite material is believed to increase differentiation of cells at the interface therebetween. The FA has is more electronegative than FHA and HA. As shown below, experiments have demonstrated that sintered FA promotes terminal differentiation of keratinocytes, and to a much higher degree than sintered FHA and HA.

The surface charge may be measured as the zeta potential. In some cases, the zeta potential of FA is more than double the zeta potential of FHA or HA sintered under the same conditions. The zeta potential of the fluoridated apatite coatings disclosed herein may be less than (e.g., have a greater negative value than) about −10 mV, such as about −10 mV to −80 mV, about −20 mV to −65 mV, about −26 mV to −80 mV, about −26 mV to −65 mV, about −40 mV to −80 mV, less than about −26 mV, less than about −35 mV, or less than about −40 mV. The inventors currently believe that the electronegativity of the fluorine atoms in the fluoridated apatite drive the zeta potential lower and stimulate cell adhesion, such as by causing differentiation.

The coated implants disclosed herein include a fluoridated apatite coating that has been sintered at a temperature between about 950° C. and 1,350° C. (e.g., about 1,050° C. to 1,250° C., about 1,050° C. to 1,150° C., or about 1,150° C. to 1,250° C.), exhibits a surface morphology that is different from unsintered fluoridated apatite (e.g., includes agglomerations of particles), and has a zeta potential that is lower than −10 mV (e.g., less than −26 mV, less than −40 mV, or about −26 mV to −65 mV).

Zeta potential measurements were used to identify the electrical potential of the material surfaces (Table 1). These data showed that synthesized apatites, were negatively charged at physiological pH (e.g., 7.4). As shown in Table 1 below, sintering appeared to increase negative zeta potential values of the HA, FHA, and FA, the FA sintered at 1250° C. produced the most negatively charged surface.

Overall surface charges of synthesized apatites (HA, FHA, and FA) were quantified using zeta potential. Four samples of each species of apatite (HA, FHA, and FA) were provided. They included an unsintered sample, a sample sintered at 1,050° C., a sample sintered at 1,150° C., and a sample sintered at 1,250° C. The zeta potential of the samples was examined using a Massively Parallel Phase Analysis Light Scattering (MP-PALS) spectrometer (Mobius mobility instrument; Wyatt Technology Corp., Santa Barbara, Calif.). For the measurement, 100 mg of the various apatite samples (powders) were suspended in a 10 mL solution of 0.154M NaCl (pH 7.4) to acquire a concentration of 0.01 g/mL. Next, 100 μL of this suspension was placed into a cuvette. After waiting a minute for the larger particles to settle to the bottom, each sample was measured nine times. The zeta potential was reported as the average value ±95% CI. Table 1 provides the results of the analysis of the zeta potential of various apatite samples, both sintered and unsintered.

TABLE 1

| Material Type | Zeta Potential (mV) |
|---|---|
| FA (unsintered) | −19.0 ± 1.7 |
| FA - sintered at 1050° C. | −15.0 ± 3.2 |
| FA - sintered at 1150° C. | −28.2 ± 9.1 |
| FA - sintered at 1250° C. | −60.4 ± 2.0 |
| FHA (unsintered) | −8.7 ± 2.9 |
| FHA - sintered at 1050° C. | −15.9 ± 5.3 |
| FHA - sintered at 1150° C. | −15.4 ± 4.0 |
| FHA - sintered at 1250° C. | −15.6 ± 5.2 |
| HA (unsintered) | −9.8 ± 2.3 |
| HA - sintered at 1050° C. | −16.1 ± 4.3 |
| HA - sintered at 1150° C. | −15.1 ± 5.1 |
| HA - sintered at 1250° C. | −24.2 ± 8.6 |

Various techniques may be used to manufacture the fluoridated apatite particles and coat the particles on implants.

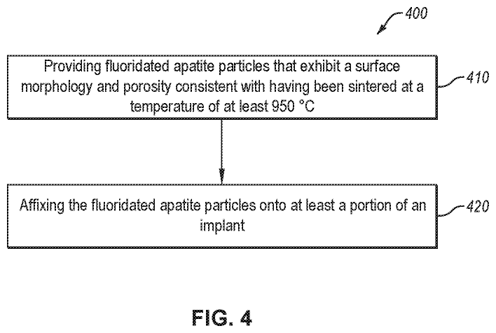

FIG. 4 is a flow diagram of a method 400 of making a coated implant, according to an embodiment. The method 400 includes block 410 of providing fluoridated apatite particles that exhibit a surface morphology and porosity consistent with having been sintered at a temperature of at least 950° C. and block 420 of affixing the fluoridated apatite particles onto at least a portion of an implant.

Block 410 of providing fluoridated apatite particles that exhibit a surface morphology and porosity consistent with having been sintered at a temperature of at least 950° C. may include providing FA particles, FHA particles, or combinations of the foregoing. Providing fluoridated apatite particles may include forming the fluoridated apatite particles into a cohesive mass such as a pellet, wafer, sheet, or other body via pressing, rolling, molding, or the like. The fluoridated apatite particles may be sintered prior to, contemporaneously with, or after affixing the fluoridated apatite particles onto the at least a portion of the implant.

In some examples, providing the fluoridated apatite particles may include providing unsintered fluoridated apatite particles and sintering the apatite particles. The fluoridated apatite particles may exhibit the surface morphology, porosity, zeta potential, average particle size, or any other characteristics of the fluoridated apatite particles disclosed herein.

Providing fluoridated apatite particles may include sintering the fluoridated apatite particles. The fluoridated apatite particles may be sintered as a loose powder or in the cohesive mass (e.g., pressed pellet of individual FA particles). Sintering the fluoridated apatite particles may include heating the fluoridated apatite particles to a temperature of at least about 950° C., such as about 950° C. to 1,350° C., about 1,050° C. to 1,250° C., about 1,050° C. to 1,150° C., about 1,150° C. to 1,250° C., at least 1,050° C., at least about 1,150° C., less than about 1,500° C., or less than about 1,250° C. The heating (e.g., sintering) may be carried out for at least 1 minute, such as about 1 minute to 24 hours, about 1 hour to 18 hours, about 2 hours to 12 hours, about 4 hours to 10 hours, about 20 minutes to 4 hours, about 30 minutes to 3 hours, about 1 hour to 10 hours, about 8 hours to about 16 hours, at least about 2 hours, less than about 24 hours, or less than about 12 hours. The above-noted sintering times may be hold times at the sintering temperature. For example, a plurality of fluoridated apatite particles may be placed in a sintering oven that is ramped up to the sintering temperature at a selected rate (e.g., about 5° C./min, about 7° C./min., about 10° C./min., about 5° C./min to 15° C./min, or about 1° C./min or more), maintains the sintering temperature for the selected duration, and ramps back down to ambient temperature at a selected rate (e.g., any of the rates disclosed above). The sintering temperatures within the ranges disclosed herein do not alter the chemical composition of the fluoridated apatites disclosed herein.

Sintering the fluoridated apatite particles may include heating the fluoridated apatite particles in an inert atmosphere (e.g., N₂ or Argon), in a vacuum, in an oxidizing atmosphere (e.g., oxygen), in an open atmosphere (e.g., in the presence of oxygen, carbon dioxide, N₂, etc.), or combinations of any of the foregoing.

In some embodiments, providing fluoridated apatite particles may include forming the fluoridated apatite particles, such as FA particles, FHA particles, or a mixture thereof. In some embodiments, a continuous aqueous precipitation method may be used to synthesize fluoridated apatite. Fluoridated apatite may be produced with a selected Ca/P ratio. For example, an FA with Ca/P ratio of 1.67 may be produced as set forth below. The FA may be synthesized under nitrogen atmosphere by mixing 250 ml of 1.2 M Ca(NO₃)₂ solution and 250 ml of 0.72 M Na₂HPO₄ solution containing stoichiometric ratios of NaF. Both Ca(NO₃)₂ and Na₂HPO₄/NaF solutions may be dispensed at a rate of 2.4 ml/min into a 12-liter reaction flask containing 10 liters of deionized water heated to a pre-selected isothermal temperature of 95° C. The reaction mixtures may be stirred at a selected speed during preparation. The stirring speed may be controlled, such as 50 rpm to 300 rpm, 100 rpm to 200 rpm, or less than 500 rpm. The pH is maintained at 9.0, such as by auto-titrating with a pH-STAT controller and AUTO burette with a 1M NaOH solution. After which, the mixture may be digested for an additional 1 hour under the same isothermal conditions to form FA. The FA may be obtained by filtering the mixture, and washing the mixture (e.g., four times) with doubly deionized water to remove all soluble byproducts (i.e., salts), and ethanol. The final residue may be dried, such as at 60° C. for 48 hours. While a specific example of forming FA is disclosed above, other techniques and reagents may be used for forming FA or FHA.

FA and FHA may be prepared by a precipitation method at an elevated temperature, such as at least 50° C. (e.g., 50° C. to 95° C.). FHA and FA may be synthesized by mixing 200 ml of 0.6M Ca(NO₃)₂ solution and 200 ml of 0.36 M Na₂ HPO₄ solution containing various concentrations of NaF at 1.4 ml/min into the 5-liter flask containing 4 liters of doubly deionized water pre-equilibrated to the target temperature. The stirring speed may be controlled such as at 200 rpm and the pH may be maintained such as at 9.0 by titration with 1M NaOH solution using a pH-STAT controller and AUTO burette. After mixing of the calcium and the phosphate/carbonate/fluoride solutions, the mixture may be digested for a selected duration, such as at least 1 hour, under the same conditions. The FA or FHA may be obtained by filtering the mixture, and may be washed one or more times (e.g., three times) with deionized water. The final residue may be oven dried, such as at 60° C. for 48 hours. The resulting unsintered solid may be ground into a fine powder and stored for later use for forming fluoridated apatite coatings. Various forms of FHA (25% to 75% of fluoride content) may be synthesized by changing the ratio of the reactants. X-ray diffraction and Fourier transform infrared spectroscopy may be used on the powders to analyze the crystallographic phase and the degree of fluorination, respectively.

The inventors have found the crystallinity and solubility properties of fluoridated apatite synthesized at 50° C. is closer to that of bone and dentin. The inventors have found the crystallinity and solubility properties of fluoridated apatite synthesized at 95° C. is closer to enamel (high crystallinity and lower solubility). Accordingly, the crystallinity and solubility of the fluoridated apatite may be selectively customized by controlling the temperature of the reaction mixture during formation of the fluoridated apatite.

The solid FA particles may be further subjected to sintering at a predetermined temperature as disclosed herein, may be ground down to acquire the desired particle sizes needed for the coating operations, and stored at room temperature in covered glass containers until further use. For example, forming the fluoridated apatite particles may include sintering the fluoridated apatite particles at a temperature of about 950° C. to about 1350° C. as disclosed herein. Such sintering may be carried out under any of the sintering conditions (e.g., inert atmosphere) for any of the sintering durations disclosed herein.

Block 420 of affixing the fluoridated apatite particles onto at least a portion of an implant may include coating the fluoridated apatite onto at least a portion of one or more surfaces of an implant body. The implant body may be similar or identical to any of the implant bodies disclosed herein in one or more aspects, such as a percutaneous implant, an osseointegrated implant, a dental implant, or the like. Various techniques may be used to affix the fluoridated apatite particles to the implant body.

In some embodiments, affixing the fluoridated apatite particles onto at least a portion of an implant may include one or more of dip coating, sputter coating, pulse layer deposition, hot pressing, isostatic pressing, electrophoretic deposition, thermal spraying, ion beam assisted deposition (“IBAD”), ultrasonic spray pyrolysis, or sol-gel techniques.

In some embodiments, affixing the fluoridated apatite particles onto at least a portion of an implant may include one of IBAD or ultrasonic spray pyrolysis. The IBAD process is a material coating technique that combines ion implantation with simultaneous sputtering or another physical vapor deposition (PVD) technique. Besides providing independent control of parameters such as ion energy, temperature and arrival rate of atomic species during deposition, this technique is especially useful to create a gradual transition between the implant body and the deposited fluoridated apatite (e.g., FA). This technique of depositing coating results in less built-in strain than by other techniques. These two properties can result in coatings with a much more durable bond to the substrate.

During IBAD deposition, material is vaporized by an electron-beam evaporator and condenses on the substrate while ions are simultaneously accelerated into the growing coating (e.g., film of FA material). This concurrent ion bombardment controls coating properties such as morphology, density, stress, crystallinity, and chemical composition. The porous, columnar microstructure observed in conventional PVD films is eliminated as energy from the ion beam increases atomic surface energy, allowing loosely bonded coating atoms to fill voids, densifying the coating. The IBAD coatings typically have nano-crystalline or amorphous microstructure, with crystal size being a predictable function of the ion beam parameters. Coating adhesion is enhanced by pre-deposition ion beam exposure, which removes contaminants and oxide layers while increasing the substrate-coating reactivity to improve chemical bonding. The IBAD process creates an intermixed zone of substrate and coating atoms, eliminating the formation of interfacial voids, improving coating adhesion. As two to three materials can be vaporized at the same time, co-deposition of FHA and FA is possible. This technique may produce relatively larger (1-2 mm) fluoridated apatite particle sizes.

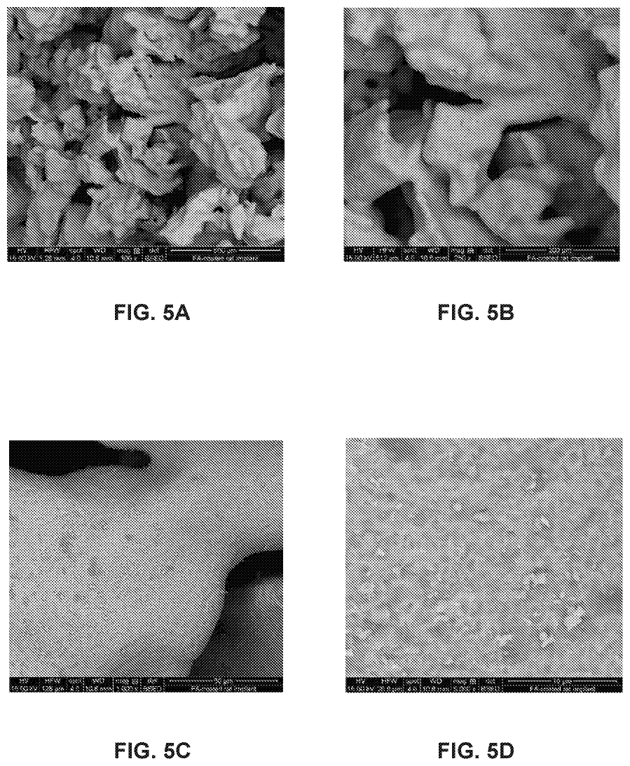

In some examples, the Iontite™ technique from N₂ Biomedical of Bedford Mass. may be used for coating the implants with fluoridated apatite (e.g., FA). It has been found that 60-200 μm (particularly 70-100 μm) bulk particle sizes of sintered fluoridated apatite were conducive for obtaining a uniform coating. The sintered FA particles were used to coat sample implants. FIGS. 5A-5D are electron photomicrographs of an FA coating on an implant. The FA coating consisted of sintered FA particles with a bulk structure measuring about 60 μm to about 200 μm. FIG. 5A is a photomicrograph of the FA coating taken at 100× magnification. FIG. 5B is a photomicrograph of the FA coating taken at 250× magnification. FIG. 5C is a photomicrograph of the FA coating taken at 1,000× magnification. FIG. 5D is a photomicrograph of the FA coating taken at 5,000× magnification. As shown, the FA particles are agglomerated into a new bulk structure (on the implant) having a greater porosity than the as-sintered FA particles and an even smoother microstructure than the as-sintered FA particles (see FIGS. 3A-3D). The bulk structure of the coating may be amorphous. The structure of the fluoridated apatite particles in the coating may be as disclosed above (e.g., the sintered agglomerations of particles) with respect to the sintered agglomerations of fluoridated apatite particles FIGS. 3A-3D. A binder or other components may be present in the coating.

In some embodiments, ultrasonic spray pyrolysis coating technique may be used to affix the coating to the implant body, because it combines both, synthesis and coating simultaneously. Ultrasonic spray pyrolysis as a material synthesis technique is simple, inexpensive, non-vacuum technique, and it has a relatively high deposition rate in a non-vacuum environment and can be easily scaled up. Fluoridated apatite (e.g., FA) particles may be produced via ultrasonic spray pyrolysis where organic and inorganic salts can be used as precursors for the preparation of the fluoridated apatite particles. The precursor solution is ultrasonically atomized before the droplet mist goes into a furnace, where thermal decomposition of the precursor material takes place and reactions of the decomposed materials may form the fluoridated apatite particles. If the implant body is located inside of the furnace, the precursor solution may be directed onto the implant body where the mist sprays onto the implant body to simultaneously form and affix the fluoridated apatite particles onto the implant body.

Coatings of different materials with different porosity, microstructure, grain size, grain orientation and possibly different composition and phase can be produced by ultrasonic spray pyrolysis by controlling the deposition variables. Possible precursors containing Ca, P, and F precursors of FA include, but are not limited to the precursors listed in Table 1 below.

TABLE 1

| Element | Precursor |
|---|---|
| Ca | Ca(CH₃COO)₂ |
| | CaFPO₃•H₂O |
| | Ca(NO₃)₂•4 H₂O |
| | Calcium oxide |
| | CaCl₂ |
| P | H₃PO₄ |
| | CaFPO₃•H₂O |
| | Triethylphosphate ((C₂H₅)₃PO₄) |
| | Tributyle phosphate |
| | Diammonium hydrogen phosphate |
| | Phosphoric pentoxide |
| | NaH₂PO₄•H₂O |
| F | CaFPO₃•H₂O |
| | Trifluoroacetic acid (CF₃CO₂H) |
| | Trifluoroacetic acid |
| | Hexafluorophosphoric acid |
| | NaF |
| | NH₄F |

Returning to FIG. 4, other coating techniques may be used, such as sol-gel dip-coating or applying an adhesive material to the implant body and adhering the fluoridated apatite particles thereon.

Affixing the fluoridated apatite particles onto at least a portion of an implant may include affixing an amount of fluoridated apatite particles effective to form a coating having any of the thicknesses, other dimensions, or locations of coatings disclosed herein.

The method 400 may include providing the implant body. The method 400 may include forming at least a portion of the implant body.

Experiments were carried out to determine the effectiveness of various embodiments of fluoridated apatite coatings for adhering to tissue cells via in-vitro experiments. To determine the extent of cell adhesion on cells that are relevant to percutaneous applications, keratinocytes were grown directly on the surface of the manufactured apatite (HA, FHA, and FA) pellets. For this assessment, HaCaT cells (T0020001; AddexBio Technologies, San Diego, Calif.) were grown in the recommended growth media until confluent.

A cell density of 1×105 cells/cm² surface area was suspended in 75 μL of culture media and carefully placed as a droplet on each pellet in individual wells of a 12-well plate. Additionally, titanium pellets (RD-128Ti; BioSurface Technologies Corporation, Bozeman, Mont.) were obtained and used as experimental controls. Before being utilized in cell or bacterial experiments, Titanium pellets were passivated, washed in 70% ethanol, and steam autoclaved. As the titanium pellets were larger than the HA/FHA/FA pellets, the cells were suspended in 150 μL of culture media to ensure the topmost face of the pellet was thoroughly seeded with cells. The samples were moved to a humidified incubator for a period of two hours to permit initial cell adhesion. Following this two-hour period, each well was slowly filled with 2 mL of fresh media, fully submerging the samples.

To count the number of adherent cells following the two-day experiment, pellets were gently washed in phosphate buffered saline (PBS), moved to a new culture plate, and incubated with 0.5 mL of 0.25% Trypsin. To inactivate the Trypsin, 0.5 mL of fresh media containing fetal bovine serum (FBS) was added to each pellet. The Trypsin/media solution was then flushed across the face of the pellet several times with a pipette in order to remove any remaining adherent cells. A hemocytometer was used to count the cells that had adhered to the surface. Data is reported as the percentage of cells counted following the two-day period relative to the initial quantity of cells seeded on each respective pellet.

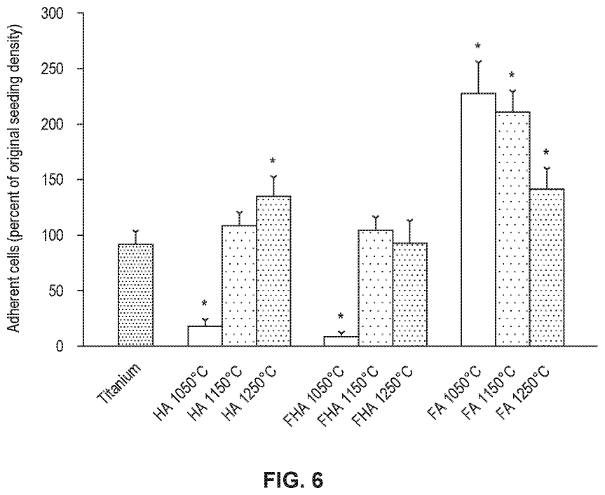

FIG. 6 is a bar chart showing the enumerated adherent HaCaT cells, from each sample surface two days after seeding. Results are presented as means±SEM. Expressed as a percentage of adherent cells relative to the original seeding density, titanium pellets had a normalized adherence rate of 92±12%. HA pellets sintered at 1050° C. had a significantly lower cell count than the titanium pellets. Conversely, HA pellets sintered at 1250° C. had significantly more adherent cells after two days compared to titanium pellets. Similar to HA pellets sintered at 1050° C., FHA pellets sintered at this temperature had a very low adherence rate. FHA pellets sintered at 1150° C. and 1250° C. had adherence rates comparable to those observed on HA pellets sintered at the same temperatures.

The largest difference in the two-day adhesion and proliferation rate of HaCaT cells was observed within the groups of FA pellets sintered at 1050° C. and 1150° C. (FIG. 5). FA pellets sintered at 1050° C. had an adherence rate of 227±28%, while FA pellets sintered at 1150° C. had an adherence rate of 210±18%. There were no statistically significant differences between these two groups; however, the adherence rate observed on these two surfaces was significantly higher than any other experimental group. FA pellets sintered at 1250° C. had a slightly lower adherence rate (143±17%) than the FA pellets sintered at the lower temperatures. Nonetheless, this group maintained a significantly higher HaCaT cell adherence over the control titanium pellets.

Keratinocytes adherence to FHA and/or FA surfaces may drive the differentiation of epithelial cells into mature, terminally differentiating phenotypes such as those of suprabasal epithelial cells, a transition highly important for preserving the integrity of the epithelial tissue attachment at the percutaneous implant interface. The extent of cell differentiation of various coatings formed at various sintering temperatures was experimentally determined.

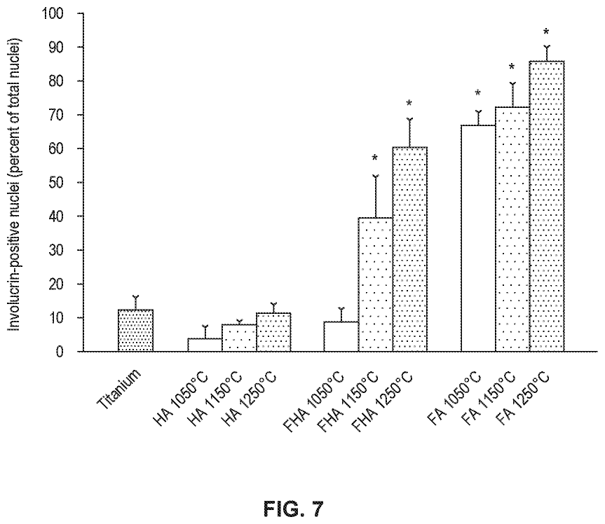

Involucrin, a marker of terminal differentiation in keratinocytes, was utilized in immunocytochemistry experiments to provide a semi-quantitative analysis of the rate of differentiation of HaCaT cells. Results from these experiments further suggested the enhanced ability of FA surfaces to promote terminal differentiation of keratinocytes. FIG. 7 is a bar chart showing the enumerated Involucrin-positive nuclei on the respective samples. Expressed as a percentage of Involucrin-positive cells relative to the total number of nuclei, keratinocyte cells cultured on FA pellets for two days were undergoing differentiation to a high degree. FA pellets sintered at 1250° C. had the highest relative levels of differentiation (86±4%), followed by FA sintered at 1150° C. (72±7%), and finally, FA sintered at 1050° C. (67±4%). This is significantly higher than either the titanium controls or any of the HA pellets, all of which had an average of approximately 10% differentiated cells. Similar to FA, FHA pellets sintered at high temperatures also induced significantly more keratinocyte differentiation than HA or titanium. Although the relative number of differentiated cells on FHA sintered at 1050° C. (8±4%) was similar to that of titanium and HA, there were significantly more Involucrin-positive cells observed on FHA pellets sintered at both 1150° C. (40±12%) and 1250° C. (60±8%).

Similar to the cell adhesion experiments described above, bacteria (S. aureus) were grown on the top face of the various pellets (HA, FHA, and FA at the sintering temps used in the above-described experiments) and the rate of adherence was observed after a predetermined period of time. The resultant colony forming units (CFUs) were normalized to the surface area available for bacterial adhesion. Although the mean adhesion and proliferation rates varied considerably amongst the experimental groups, no significant differences were observed within or between these groups. Thus, the experiment did not reveal any statistically significant differences between any of the fluoridated surfaces and titanium for hosting bacteria. Accordingly, the inventors believe tissue cells can be preferentially bonded to the fluoridated apatites disclosed herein without increasing bacterial adhesion.

As demonstrated herein, fluoridated apatite coated surfaces (especially FA surfaces) sintered at 1,050° C. to 1,250° C. (and more particularly 1,050° C. to 1,150° C.), are more conducive to keratinocyte adhesion and differentiation when compared to titanium surfaces and HA surfaces.

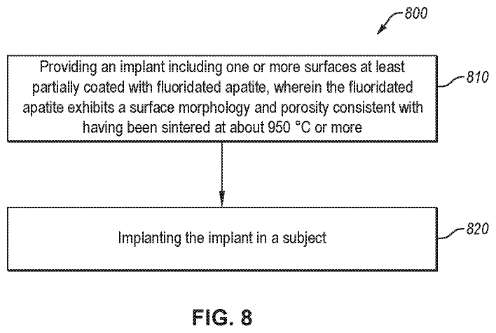

FIG. 8 is a flow chart of a method 800 of using an implant having a fluoridated apatite coating, according to an embodiment. The method 800 includes the block 810 of providing an implant including one or more surfaces at least partially coated with fluoridated apatite, wherein the fluoridated apatite exhibits a surface morphology and porosity consistent with having been sintered at about 950° C. or more and the block 820 of implanting the implant in a subject.

The block 810 of providing an implant including one or more surfaces at least partially coated with fluoridated apatite, wherein the fluoridated apatite exhibits a surface morphology and porosity consistent with having been sintered at about 950° C. or more may include providing any of the implants disclosed herein with any of the coatings disclosed herein on one or more surfaces thereof. The implant may be a percutaneous implant, an osseointegrated implant, a dental implant, or combinations thereof. Providing an implant may include making an implant, such as by using any of the techniques disclosed herein. For example, providing an implant may include coating an implant body with fluoridated apatite particles, such as by any of the techniques disclosed herein.

The fluoridated apatite of the implant may include fluorapatite particles, fluorohydroxyapatite particles, or a mixture thereof, such as any of the fluoridated apatite particles disclosed herein (e.g., having any of the characteristics or properties disclosed herein). For example, the fluoridated apatite particles may have any of the zeta potentials, surface morphologies or porosities disclosed herein. The fluoridated apatite may be disposed on the implant in any of the thicknesses or distributions disclosed herein.

The implant may include a titanium body. The implant may be sized and shaped as a percutaneous implant, an osseointegrated implant, a dental implant, or the like.

The block 820 of implanting the implant in a subject may include implanting (e.g., surgically) the implant into the tissue of a subject, such as into the skin, bone, or other tissues of a subject. Implanting the implant in a subject may include positioning the implant within the subject such that the fluoridated apatite coating therein is placed in contact with one or more of skin, bone, or tissues of the subject. For example, an osseointegrated implant with a fluoridated apatite coating (e.g., FA) disposed along a length thereof may be inserted into bone and protrude through the skin of the subject whereby the fluoridated apatite coating contacts one or both of the bone or the skin of the subject. The coating may be disposed on the implant such that the coating portion of the implant extends out of the skin of the subject, in some embodiments.

The method 800 may include cleaning the implantation site, such as cleaning the interface between the skin of the subject and the implant. Such cleaning may include washing with a water or another fluid (e.g., iodine, soap, alcohol, etc.). The method 800 may include removing the implant.

### Working Examples

In vivo tests were carried out utilizing the implants having the fluoridated apatites disclosed herein.

Titanium, HA, FHA, and FA pellets were formed for implantation into rats. CD Hairless rats (Charles River Labs) were each implanted with individual titanium, HA, FHA, or FA pellets and run for 12 weeks, at which point they were sacrificed. Gross inspection revealed no obvious signs of host rejection of any pellet composition. The skin surrounding each pellet was harvested and sectioned for histology analysis. Tissue sections were then utilized to measure the respective thickness of the fibrous capsules that had formed around the pellets.

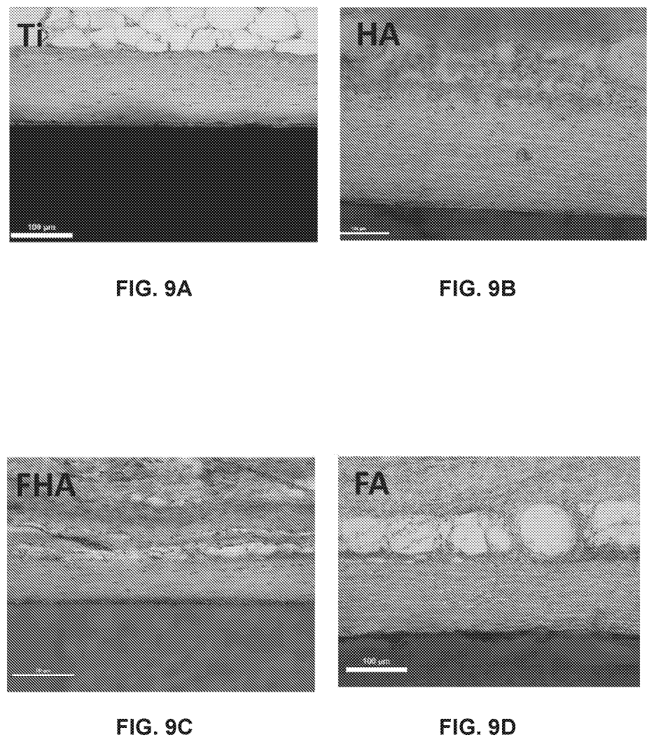

FIGS. 9A-9D are photographs of tissue sections for histology analysis of implanted titanium, HA, FFA, and FA pellets, according to embodiments. FIGS. 9A-9D show capsule thickness in rat tissues surrounding Titanium, HA, FHA, and FA pellets. FIG. 9A shows capsule thickness in rat tissue surrounding Titanium pellets. FIG. 9B shows capsule thickness in rat tissue surrounding hydroxyapatite pellets. FIG. 9C shows capsule thickness in rat tissue surrounding fluorohydroxyapatite pellets. FIG. 9D shows capsule thickness in rat tissue surrounding fluorapatite pellets. The analysis revealed that FHA pellets exhibited the least fibrous capsulation, having the thinnest area of fibrous tissue, as shown by the tissue section below the lightest portion in FIGS. 9A-9D.

In vivo tests were carried out utilizing the implants having the fluoridated apatites disclosed herein. Raw HA, FHA, and FA powders were sintered in a high-temperature furnace at the 1050° C., 1150° C., and 1250° C. Next, the sintered powders were crushed with mortar and pestle to break up the aggregates that form as a result of sintering. Finally, the powders were separated by particle size using a sieve with several different screens. Particles of selected size (60 μm to 200 μm) were coated on a titanium rat implant via the Iontite™ technique from N₂ Biomedical of Bedford Mass. Over 90% of the visible surface area of the titanium implant was covered by a homogenous layer of the HA, FHA, or FA. Control samples of uncoated implants (titanium) were also utilized.

A total of twenty rats underwent implantation surgery. These rats each received a single implant along their dorsum. The groups included Group 1 (control; uncoated titanium implant), Group 2 (implant coated with HA sintered at 1150° C.), Group 3 (implant coated with FHA sintered at 1150° C.), and Group 4 (implant coated with FA sintered at 1150° C.).

During implantation, anesthetized animals were placed in the ventral recumbent position and a small incision was made parallel the spinal column. Blunt-tipped scissors were then utilized to create a pocket under the skin on the opposite side of the spine. A stoma was created over this pocket using a biopsy punch slightly larger in diameter than the percutaneous post of the implant. The implant was placed in the subdermal pocket and the post protruded through the stoma. Following surgery, the incision line was sutured closed and the wounds were covered with a Tegaderm transparent, breathable membrane. The animals were observed daily for signs of clinical infection and general health and well-being. No signs or symptoms of implant rejection were observed during the study period, and the animals were sacrificed 8 weeks post-surgery. Tissues were then collected for general histology, immunohistochemistry (IHC), and qRT-PCR experiments.

The skin and implant samples from the rats were embedded in PMMA to preserve the soft tissue interface in contact with the various implants. After embedding the samples, they were sectioned, ground and polished, and then stained with Hematoxylin and Eosin (H&E) for general histology. Using this technique, it was possible to examine epithelial downgrowth on each of the samples.

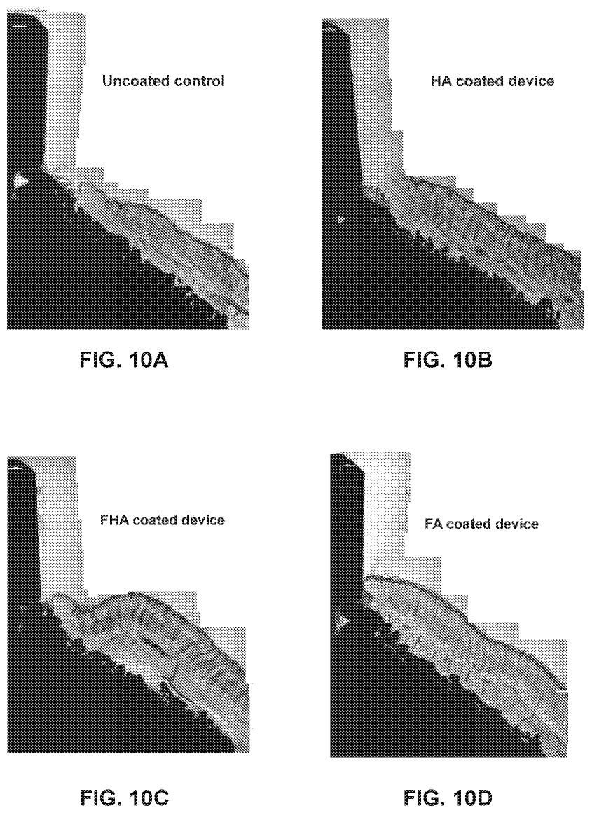

FIGS. 10A-10D are photos of stained sections of samples showing epithelial downgrowth along the implant surfaces, according to embodiments. In FIGS. 10A-10D, H&E stained sections showing epithelial (light grey) downgrowth along the implant surface (black). Percutaneous post of the implants are vertically aligned and subdermal barrier is angled portion of visible implants in FIGS. 10A-10D. FIG. 10A is a photo of an H&E stained section showing epithelial downgrowth along the implant surface of an uncoated titanium implant of Group 1. FIG. 10B is a photo of an H&E stained section showing epithelial downgrowth along the implant surface of an HA coated titanium implant of Group 2. FIG. 10C is a photo of an H&E stained section showing epithelial downgrowth along the implant surface of an uncoated titanium implant of Group 3. FIG. 10A is a photo of an H&E stained section showing epithelial downgrowth along the implant surface of an uncoated titanium implant of Group 4. As shown in FIGS. 10A-10D, FHA and FA-coated implants of Groups 3 and 4 exhibited the least downgrowth, with the FA-coated implants of Group 4 exhibiting almost no downgrowth. The FA and FHA mitigated much of the downgrowth observed in the HA coated implant of Group 3 (FIG. 10B) and uncoated titanium controls of Group 4 (FIG. 10A).

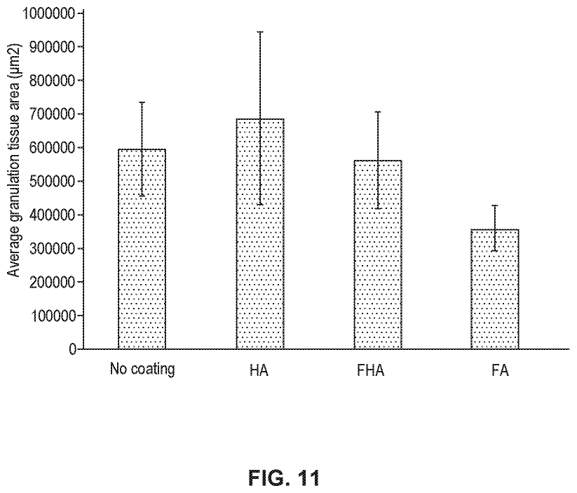

The average granulation tissue area for each sample of Groups 1-4 were measured and compared. FIG. 11 is a graph of quantitative values for epithelial downgrowth observed along the various implants of Groups 1-4, according to embodiments. As shown in FIG. 11, the FA coated sample of Group 4 had the lowest average granulation tissue area which correlates to the least epithelial downgrowth of the samples. The FHA coated sample of Group 3 had a lower average granulation tissue area than the uncoated titanium implant of Group 1. The HA coated sample of Group 2 had the highest average granulation tissue area of Groups 1-4, which correlates to the highest average epithelial downgrowth.

In vivo tests were carried out on pigs. A total of six implants were placed along the back of each individual animal. One implant per animal was an uncoated titanium control implant, and the remaining titanium implants were coated with Commercial HA (from Himed; Old Bethpage, N.Y.), HA sintered at 1150° C., FHA sintered at 1150° C., FHA sintered at 1250° C., and FA sintered at 1150° C. The surgical protocol is described below.

After an aseptic surgery preparation, the cranial (superior) and caudal (inferior) boundaries of the surgical field were marked, as well as staggered percutaneous post exit-sites. Three bilateral, semicircular subcutaneous pockets were incised by blunt dissection from a single dorsal midline incision. Percutaneous implant exit sites were created using a 7 mm biopsy punch. The implant type and dorsal implantation site (e.g., relative medial-lateral and cranial-caudal position) were determined randomly for the first animal and all subsequent animals had an identical implant type arrangement. Following implantation, the incision line was closed, the entire surgical field was dressed with sterile gauze, and the animals were allowed to ambulate freely for the remainder of the study. Animals were separated into three groups based on their recovery time following surgery before they were sacrificed. Group A was examined at 4 weeks and comprised 5 animals, Group B was examined at 8 weeks and comprised 5 animals, and Group C was examined at 16 weeks and included 5 animals. Sutures were removed 1-2 weeks following surgery and the wound dressings were changed a minimum of once weekly throughout the duration of the experiment. Upon sacrifice, the implant sites and adjacent tissues were removed for histological staining, immunohistochemistry (IHC) analyses, and RNA sequencing.

The implant and remaining adherent soft tissues were fixed in formalin and embedded in PMMA as described in previous reports. All of the samples from this experiment have now been embedded, processed, and stained for histological analyses. Surprisingly, negligent epithelial downgrowth was observed on all of the samples, regardless of the coating. This was surprising, as downgrowth has commonly been observed in this model when utilized in the past. However, upon examination, it was readily apparent that the animals had put on a significant amount of new tissue mass (e.g., fat, skin thickness, etc.) post-surgery. It was likely that the new growth outpaced any downgrowth, thereby “masking” the downgrowth that was likely still occurring. Such growth was likely due to the relatively young (3-4 months old at surgery) animals.

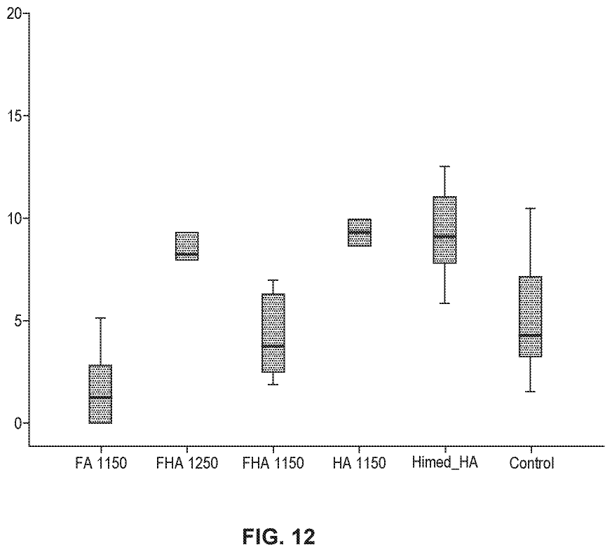

Granulation tissue was relatively abundant in various samples at the skin-device interface. FIG. 12 is a graph of the granulation tissue area observed on the various implant surfaces, according to an embodiment. The FA-coated implant (FA1150) had the lowest mean granulation tissue area, which was statistically significant compared to the uncoated titanium control (Control) and both the commercial (HimedHA) and synthesized HA-coated (HA1150) implants. This suggests that the FA-coated devices elicited an earlier healing response, moving the stoma out of an acute wound healing phase and towards a more mature “healed” phenotype. The FHA-coated implant (FHA1150) exhibited a granulation tissue area similar to that of the uncoated titanium control.

While various aspects and embodiments have been disclosed herein, other aspects and embodiments are contemplated. The various aspects and embodiments disclosed herein are for purposes of illustration and are not intended to be limiting.

1. An implant, comprising:
an implant body defining one or more surfaces; and
a fluoridated apatite coating disposed on at least a portion of the one or more surfaces;
wherein the fluoridated apatite coating exhibits a surface morphology and porosity consistent with having been sintered at about 950° C. to about 1350° C.

2. The implant of claim 1, wherein the implant body includes a percutaneous implant and the at least a portion of the one or more surfaces includes portions of the implant body expected to be disposed in contact with a tissue of a subject upon implantation.

3. The implant of claim 1, wherein the implant body includes an osseointegrated implant or a dental implant.

4. (canceled)

5. The implant of claim 1, wherein the fluoridated apatite coating exhibits a surface morphology and porosity consistent with having been sintered at about 1050° C. to about 1250° C.

6. The implant of claim 1, wherein the surface morphology includes spherical, semi-spherical, prismatic, pseudo-prismatic, ellipsoid, or irregularly rounded fluoridated apatite particles that are devoid of rod-like or needle-like fluoridated apatite particles.

7. The implant of claim 1, wherein the fluoridated apatite coating includes fluorapatite particles, fluorohydroxyapatite particles, or a mixture thereof.

8. The implant of claim 1, wherein the fluoridated apatite coating exhibits a zeta potential of less than −10 mV.

9. The implant of claim 1, wherein the fluoridated apatite coating exhibits a zeta potential of about −26 mV to −80 mV.

10. The implant of claim 1, wherein the implant body includes titanium.

11. The implant of claim 1, wherein the fluoridated apatite coating has a thickness of about 1 μm to about 1 mm.

12. A method of forming a coated implant, the method comprising:
providing fluoridated apatite particles that exhibit a surface morphology and porosity consistent with having been sintered at a temperature of about 950° C. to about 1350° C.; and
affixing the fluoridated apatite particles onto at least a portion of an implant.

13. (canceled)

14. (canceled)

15. (canceled)

16. (canceled)

17. (canceled)

18. (canceled)

19. The method of claim 12, further comprising sintering the fluoridated apatite particles in ambient air.

20. The method of claim 12, further comprising sintering the fluoridated apatite particles in an inert atmosphere.

21. The method of claim 12, wherein providing fluoridated apatite particles includes providing fluorapatite particles with an average particles size between 60 μm to 200 μm.

22. The method of claim 12, wherein providing fluoridated apatite particles includes providing fluoridated apatite particles having a zeta potential of less than −10 mV.

23. (canceled)

24. The method of claim 12, wherein affixing the fluoridated apatite particles onto at least a portion of an implant includes using one or more of dip coating, sputter coating, pulse layer deposition, hot pressing, isostatic pressing, electrophoretic deposition, thermal spraying, ion beam assisted deposition (“IBAD”), ultrasonic spray pyrolysis, or sol-gel techniques to affix the fluoridated apatite particles to at least a portion of one or more surfaces of the implant.

25. The method of claim 12, wherein affixing the fluoridated apatite particles onto at least a portion of an implant includes affixing the fluoridated apatite particles onto a percutaneous implant, an osseointegrated implant, or a dental implant.

26. (canceled)

27. (canceled)

28. A method of using an implant having a fluoridated apatite coating, the method comprising:
providing an implant including one or more surfaces at least partially coated with fluoridated apatite, wherein the fluoridated apatite exhibits a surface morphology and porosity consistent with having been sintered at about 950° C. to about 1350° C.; and
implanting the implant in a subject.

29. (canceled)

30. (canceled)

31. (canceled)

32. (canceled)

33. The method of claim 28, wherein the fluoridated apatite exhibits a zeta potential of less than −10 mV.

34. (canceled)

35. (canceled)

36. (canceled)

37. The method of claim 28, wherein the implant includes a titanium body at least partially coated in the fluoridated apatite.

38. (canceled)

39. (canceled)

40. (canceled)

41. (canceled)

42. (canceled)

43. The method of claim 28, wherein implanting the implant in a subject includes implanting the implant such that portions of the implant having the coating thereon are in contact with one or more of skin, bone, or tissue of the subject.

44. (canceled)
